(19) United States
(12) Patent Application Publication
(10) Pub. No.: US 2005/0118147 A1
(43) Pub. Date: Jun. 2, 2005

(54) Method of using mesenchymal stromal cells to increase engraftment

(76) Inventor: Il-Hoan Oh, Seoul (KR)

Correspondence Address: Joseph Hyosuk Kim, Ph.D.;JHK Law, P.O. Box 1078, La Canada, CA, 91012-1078, US

(21) Appl. No.: 10/991,716

(22) Filed: Nov. 17, 2004

Related U.S. Application Data
(60) Provisional application No. 60/520,746, filed on Nov. 17, 2003.

(51) Int. Cl. A61K 45/00; C12N 5/08

(52) U.S. Cl. 424/93.7; 435/372

(57) ABSTRACT

The present application discloses a composition that includes a sample of donor stem cells that are desired to be engrafted to a subject; and a sample of mesenchymal stromal cells.

### BACKGROUND OF THE INVENTION

1. Field of the Invention

The present invention relates to a composition for enhancing engraftment after cotransplantation with mesenchymal stromal cells and donor stem cells from multiple stem cell sources. The invention also relates to decreasing graft versus graft rejection by cotransplantation with multiple unit donor stem cells and mesenchymal stromal cells. The invention also relates to methods of enhancing engraftment by the cotransplantation process. The present application also relates to treating, reducing or suppressing graft versus host disease by cotransplanting the donor stem cells (single or multiple units) with the mesenchymal stromal cells.

2. General Background and State of the Art

Umbilical cord blood (UCB) is an attractive source of hematopoietic stem cells (HSCs), and has many advantages over bone marrow stem cells including a higher frequency of transplantable HSCs and a higher output of progenitor cells from equivalent numbers of HSCs. In addition, public banking of UCB is creating increased accessibilty¹⁻³. Accumulating clinical evidence demonstrates that the number of total nucleated cells in a given UCB unit is the single most important parameter for successful outcome, with low cell numbers frequently resulting in delayed or failed engraftment⁴⁻⁵. Although ex vivo expansion of cord blood cells has been explored as a method to increase the input cell number at the time of transplantation, loss of engraftment has been frequently observed⁶⁻⁸, and an efficient and reliable method of expansion has yet to be determined⁹⁻¹¹.

Interestingly, immune cells in UCB are functionally immature and a lower incidence of severe graft vs. host disease (GVHD) has been observed with UCB transplantation¹²⁻¹⁴. It may be possible, therefore, to transplant UCB cells with greater HLA disparities, or to transplant multiple UCB units as a mixture to increase the absolute number of HSCs in the graft. However, several clinical trials evaluating transplantation of multi-donor UCB grafts have shown that cells from one particular donor tend to predominate over the other (or others) in the reconstitution of such patients, posing another challenge to this strategy¹⁵⁻¹⁸.

The origin of the unequal contribution of the two UCB sources toward hematopoietic reconstitution is not yet clear, and whether it is due to an unequal content of HSCs in each UCB unit or whether it occurs as a result of competition between the two grafts during the engrafting process remains to be determined. However, it has been difficult to address this issue in clinical trials due to the lack of matched single UCB transplantation groups. Furthermore, it has not yet become clear whether the increase in cell numbers created by multiple cord transplantation indeed results in a higher overall level of engraftment as compared to conventional single UCB transplantation.

To study the kinetics and the quantitative aspects of HSC engraftment, surrogate in vivo xenogeneic transplantation models have been employed, including transplantation into severe combined immunodeficiency (SCID) mice¹⁹, nonobese diabetic-SCID (NOD/SCID) mice²⁰, preimmune fetal sheep²¹, along with autologous transplantation models in large animals such as non-human primates²². The engrafting human HSCs in NOD/SCID or SCID mice have been defined as SCID-repopulating cells (SRCs) or competitive repopulating units (CRUs)¹⁹, which are the cells that can give rise to long-term repopulation and multi-lineage differentiation without exhaustion²³ in the transplanted recipients.

In the current study, using a series of HLA disparity-based combinations of UCB units transplanted into NOD/SCID mice, we show that the one-donor predominance phenomenon in double cord transplantation is not due to absence or lack of CRU content in the non-dominant graft, but rather, it occurs during the engrafting process independent of HLA disparities.

Thus, there is a need in the art to increase engraftment by cotransplanting stem cells from multiple stem cell source.

### SUMMARY OF THE INVENTION

Cotransplantation of culture-expanded mesenchymal stromal cells (MSCs) from third-party bone marrow can alleviate one-donor predominance and thereby lead to additive coengraftment resulting in higher levels of overall engraftment after multiple stem cell source transplantation.

The present invention is directed to a composition that includes: (i) a sample of donor stem cells that are desired to be engrafted to a subject; and (ii) a sample of mesenchymal stromal cells. The sample in (i) may be bone marrow stem cells, peripheral blood stem cells, or umbilical cord blood stem cells or a mixture thereof. Further, the sample of stem cells of (i) may be a mixture of a plurality of allogeneic samples. In addition, the stems cells in (i) may differ in HLA type with respect to each other. In the composition, the mesenchymal stromal cells may differ in HLA type with respect to the stem cells of (i).

In particular, the mesenchymal stromal cell sample may be present in an amount effective to reduce one-donor predominance in a subject.

In another aspect, the invention is directed to a stock composition of mesenchymal stromal cells. These cells may be cryopreserved.

In another aspect, the invention is directed to a method of decreasing one-donor predominance of stem cells in a subject, which includes administering: (i) a plurality of samples of donor stem cells; and (ii) one-donor predominance reducing effective amount of mesenchymal stromal cells to the subject thereof. In this method, the donor stem cells of (i) may be allogeneic with respect to each other. Further, the donor stem cells of (i) may be xenogeneic with respect to each other. Further in this method, the mesenchymal stromal cells may be co-transplanted with the donor stem cells. In the method above, the mesenchymal stromal cells may be administered before the donor stem cells are administered, or they may be administered after the donor stem cells are administered. In this method, the predominance may be reduced to about 0.5 to 3 for donor stem cells with respect to each other. In this method, the subject may be a mammal, including a human.

In another aspect, the invention is directed to a method for increasing engraftment of donor stem cells in a subject comprising administering: (i) a plurality of samples of donor stem cells; and (ii) engraftment effective amount of mesenchymal stromal cells to the subject thereof. In this method, the donor stem cells of (i) may be allogeneic with respect to each other. Further, the donor stem cells of (i) may be xenogeneic with respect to each other. Further in this method, the mesenchymal stromal cells may be co-transplanted with the donor stem cells. In the method above, the mesenchymal stromal cells may be administered before the donor stem cells are administered, or they may be administered after the donor stem cells are administered. In this method, the predominance may be reduced to about 0.5 to 3 for donor stem cells with respect to each other. In this method, the subject may be a mammal, including a human.

The invention is also directed to a method for suppressing graft versus host disease comprising administering: (i) a sample of donor stem cells; and (ii) engraftment effective amount of mesenchymal stromal cells to the subject thereof.

Further, the mesenchymal stromal cell as described in any of the methods above may be derived from third party bone marrow or umbilical cord blood.

In still another aspect, the invention is directed to instructions for carrying out stem cell transplantation, which comprises information carrying out the methods as described above.

In one embodiment of the invention, because of the increased engraftment results obtained using the cotransplantation method of the invention, efficacious outcome of stem cell transplantation in a subject is expected.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIGS. 1A-1C show one-donor predominant engraftment after transplantation of two UCBs as a mixture of total mononuclear cells.

A. Donor origin of cord blood cells engrafted in NOD/SCID mice as determined by PCR-SSOP. Combinations of two UCB units were made with varying degrees of HLA disparity, and total MNCs corresponding to 5×10⁴ CD34+ cells from each unit were transplanted into irradiated NOD/SCID mice as single (CH1-CH4) or double (CH1+2, CH3+4) transplants. Genomic DNA harvested from recipients' bone marrow were subjected to PCR amplification by DPB1 locus-specific primers and hybridized to allele-specific probes (P1 to P4), where positive controls were DNA from donor cells (cont1 to cont4). Shown are the results from two cohorts of double transplants with either a full match at six-loci (HLA-A, B, DR) (left) or two mismatches (HLA-B, DR) (right).

B. Profiles of RQ-PCR for the human STR to determine donor ratio of engrafted cells. Genomic DNA was harvested from mice bone marrow and subjected to real-time quantitative PCR analysis on 16 human STR markers. Shown are the results from a representative marker in each cohort. CB-A and CB-B were artificially nominated for the dominating and non-dominating cord unit, respectively.

C. Absence of additive engraftment in transplantation of two UCB units as a mixture of total mononuclear cells. Overall engraftment levels of transplanted cord blood cells in NOD/SCID mice were measured by staining harvested mice bone marrow with human cell specific anti-CD45/CD71 as described in Example 1—Materials and Methods. Shown are the mean engraftment levels from single or double cord transplantation (n=22) from nine cohorts.

FIGS. 2A-2B show effects of lineage depletion on one-donor predominance.

A. Two sets of double cord transplants were performed with lineage depleted cord blood pairs having five or six mismatches. Shown are results as analyzed by PCR-SSOP for the DRB1 locus using allele-specific probes (DR5 for CB 22 and CB32, DR4 for CB 40), where CB22, 40, or 32 represent single cord transplantation and 22+40 or 32+40 represent double cord transplantation; numbers below represent numbers of mice (n=6).

B. Quantitative analysis of engraftment after lineage depleted double cord transplantatation. Two additional cohorts of double cord transplants were analyzed for donor distribution of engrafted cells by RQ-STR and the relative ratio of engraftment levels of double over single unit transplantation (double/single engraft ratio) calculated. Lineages of donor cells were determined by % of lymphoid (CD19/20), myeloid (CD15/66b) and erythroid (Glycophorin-A) cells among the total human cells engrafted (CD45/71) (n=11 for single, 10 for double transplants).

FIGS. 3A-3C show suppression of one-donor predominance by cotransplantation of MSC from third-party bone marrow.

A. Effect of MSC cotransplantation on donor distribution as analyzed by PCR-SSOP. Total MNCs equivalent to 3×10⁴ CD34+ cells from each UCB unit were transplanted into NOD/SCID mice as either single (CM1-CM4) or mixed (CM1+2, CM3+4) transplants as described, except that 4×10⁴ MSCs were coinfused into each recipient. The donor origin of the engrafted cells was identified by PCR on the HLA-DR locus followed by hybridization to allele-specific probes (R1 for CM1, and R5 for CM2, R11 for CM3, and R6 for CM4, respectively). Shown are the results of two experiments using pairs of 5-mismatch UCBs.

B. MSC-mediated coengraftment as assessed by RQ-STR. Shown are the profiles for donor distribution analyzed by RQ-PCR on representative STR markers with percent reconstitution of dominant donor cells artificially named as donor A.

C. Increase in overall engraftment in MSC co-transplanted double cord transplantation over single unit transplantation. Total engraftment of human cord blood cells was measured by anti-human CD45/71 as described. Shown are the engraftment levels of single or double cord transplants (each n=8) in cohorts including one three-mismatch pair, two five-mismatch pairs, and one full-mismatch pair.

FIGS. 4A-4C show cotransplantation of MSCs may result in higher engraftment in double cord blood transplantation due to alleviation of donor-deviated engraftment.

A. Total engraftment of cord blood cells achieved by double cord transplants in the presence or absence of MSC cotransplantation. Multiple independent seven cohorts of double cord transplants were performed by transplanting total mononuclear cells equivalent to 3×10⁴ CD34⁺ cells for each UCB unit in the presence (n=19) and absence (n=26) of 4×10⁴ MSCs. Shown are the mean engraftment levels ±SEM of human cord blood cells in NOD/SCID mice.

B. Cumulative measurement of the extent of donor deviation in double cord engraftment in the presence or absence of MSC cotransplantation. Shown are the average percent (from type 1 and type 11) donor cell distribution and donor cell ratios from the same cohorts (a), as measured by RQ-STR.

C. Lineage distribution of human cells engrafted in the NOD/SCID mice in the presence or absence of MSC cotransplantation. Shown are the mean % of the total human cell engraftment (CD45/71) of each lineage (with SEM, n=8 each).

FIG. 5 shows schematic model for MSC-mediated coengraftment of two allogenic cord blood cells. The UCB units contain both primitive HSCs and differentiated cells including lymphocytes. During the early phase of engraftment, the allogenic immune responses by innate lymphocytes (lymphocytes contained in the graft) may be suppressed by co-transplanted MSCs due to the MSC's inhibitory effects on lymphocytes. Primitive HSCs are therefore protected from the alloimmune responses, and thus surviving HSCs induce tolerance to cells matched to their own genotypes. Therefore, although coinfused MSCs do not home to bone marrow and do not exist throughout the period of marrow reconstitution, mixed chimerism established during the early phase of engraftment with MSCs may be maintained for longer periods of engraftments.

### DETAILED DESCRIPTION OF THE PREFERRED EMBODIMENTS

In the present application, “a” and “an” are used to refer to both single and a plurality of objects.

Before the present invention and methods for using same are described, it is to be understood that the terminology used herein is for the purpose of describing particular embodiments only and is not intended to be limiting.

Definitions

As used herein, “CRU (competitive repopulating unit)” refers to a unit to measure long-term engrafting stem cells, which can be detected after in-vivo transplantations.

As used herein, “effective amount” is an amount sufficient to effect beneficial or desired clinical or biochemical results. An effective amount can be administered one or more times. For purposes of this invention, an effective amount is the amount of mesenchymal stromal cells or combination of donor stem cells and mesenchymal stromal cells that may be administered to effect beneficial engraftment of the stem cells.

As used herein, “engraftment” and “in vivo regeneration” refer to the biological phenomenon in which implanted or transplanted stem cells produce differentiated cell progeny as well as themselves in the body, and/or replace lost or damaged cells with injected cells.

As used herein, “mammal” for purposes of treatment refers to any animal classified as a mammal, including humans, domestic and farm animals, and zoo, sports, or pet animals, such as dogs, cats, cattle, horses, sheep, pigs, and so on. Preferably, the mammal is human.

As used herein, “mesenchymal stromal cells” and “mesenchymal stem cells“may be used interchangeably. The cells may be freshly prepared or may be stored prior to use. For instance, the mesenchymal stromal cells may be cryopreserved such as in liquid nitrogen or in about −70° C. The mesenchymal stromal cells may be pretreated with a chemical or radiation prior to use or storage in order to lengthen the lifetime of the cells or generally to provide some advantage to the cells. In one aspect, such preserved and optimized cells that are stored and used are the subject of the invention. Such mesenchymal stromal cells may be stored in a hospital, clinic or blood bank to be used later during administration to a subject undergoing stem cell transplantation.

As used herein, “sample” or “biological sample” is referred to in its broadest sense, and includes any biological sample obtained from an individual, body fluid, cell line, tissue culture, or other source which may contain stem cells, depending on the type of assay that is to be performed.

As used herein, “stem cell” refers to a cell with capability of multi-lineage differentiation and self-renewal, as well as the capability to regenerate tissue. Although stem cells are described mostly with respect to using umbilical cord blood stem cells in the present application, the invention is not limited to such and may include stem cells of other origin, including but not limited to liver stem cells, pancreatic stem cells, neuronal stem cells, bone marrow stem cells, peripheral blood stem cells, umbilical cord blood stem cells or a mixture thereof. Further, the invention is not limited to transplantation of any particular stem cell obtained from any particular source, but may include stem cells from “multiple stem cell sources” in mixture with one another. Thus, mesenchymal stromal cells may be used in cotransplantation of the stem cells obtained from single or multiple stem cell sources to increase the amount of engraftment.

Through effectiveness of the engraftment, the invention may be used to treat various diseases for which infusion and engraftment of stem cells would aid in treating the disease. Such diseases may include without limitation leukemia, breast cancer, lymphoma, Hodgkin's Disease, Aplastic Anemia, Sickle Cell Anemia, various other cancers, blood diseases, hereditary/genetic conditions and immune system disorders, lung cancer, Multiple Sclerosis, Lupus, AIDS and many other genetic diorders.

In addition, the stem cells may be natural stem cells or may have engineered in them various genes that do not negatively alter the effectiveness of the cells in engrafting. The cells may also be cultured before transplanting in order to maintain their viability.

As used herein, “subject” is a vertebrate, preferably a mammal, more preferably a human.

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” refers to both therapeutic treatment and prophylactic or preventative measures. Those in need of treatment include those already with the disorder as well as those in which the disorder is to be prevented. “Palliating” a disease means that the extent and/or undesirable clinical manifestations of a disease state are lessened and/or the time course of the progression is slowed or lengthened, as compared to a situation without treatment. Typically, the “treatment” entails administering additively effective stem cells to the patient to regenerate tissue.

Graft Versus Host Disease

The present invention is directed to treating, reducing or suppressing graft versus host disease by suppressing in vivo immune reaction when mesenchymal stromal cells are cotransplanted with the donor stem cells that may be a single unit donor stem cells or multiple unit donor cells from a variety of sources.

Graft-versus-host disease (GVHD) is a possible complication of any stem cell transplant that uses stem cells from either a related or an unrelated donor (an allogeneic transplant). To understand GVHD, it is helpful to compare it to a more familiar concept: rejection following a solid organ transplant.

In a stem cell transplant, the transplanted cells recreate the donor's immune system in the body of the recipient. GVHD is the term used when this donated immune system (the graft) begins to attack the recipient's body (the host)—the host's body already has damaged immune system.

There are two kinds of GVHD: acute and chronic. Acute GVHD appears within the first three months following transplantation. Signs of acute GVHD include a reddish skin rash on the hands and feet that may spread and become more severe, with peeling or blistering skin. Chronic GVHD is ranked by doctors based on its severity: stage (or grade) 1 is mild, stage (or grade) 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines.

In only two kinds of stem cell transplants are the donor and recipient 100 percent matched. One such instance is when the donor and recipient are identical twins, and the other case is when the donor and the recipient are the same person (an autologous transplant). In all other types of stem cell transplants, the donor's immune system is a very good, but not perfect, match with the recipient's immune system.

Even when transplant doctors talk of a “perfect match” between a donor and a patient, they are only saying that a handful of key immune system characteristics or “markers” are matched up. These matched markers are the most important markers for transplantation purposes, but several other, less significant markers may remain unmatched.

It is these small differences that can lead to GVHD. While a donor's immune system is establishing itself in the recipient's body, T-cells in the transplanted stem cell graft may begin to attack the recipient's body. GVHD is thought to occur when there is enough of a difference between the donor and recipient that the T-cells from the donor determine that the recipient's body is foreign.

In the present invention, by cotransplanting the mesenchymal stromal cells with either a single unit or multiple unit of donor stem cells, the immune response of the host is decreased. Therefore, the present invention may be used to reduce or prevent graft versus host disease. The donor stem cells and the cotransplanting mesenchymal stromal cells may be obtained from a variety of sources, including but not limited to umbilical cord blood or bone marrow.

Cotransplantation and Engraftment Using Umbilical Cord Blood

The present inventive system may be used to enhance graft-versus-graft tolerance. Allogeneic donor stem cells from different sources may be combined and their immunogenic tolerance to each other may be enhanced by cotransplantation with mesenchymal stromal cells. The donor stem cells and the cotransplanting mesenchymal stromal cells may be obtained from a variety of sources, including but not limited to umbilical cord blood or bone marrow.

In UCB transplantation, total cell number has been a major limiting factor, with a lower input cell number correlating with higher rates of delayed or failed engraftment^{4,5,42}. Although increasing total input cell numbers by admixing multi-donor derived UCB has been employed as an attractive strategy to overcome this limit, clinical studies have shown variable degrees of one-donor predominance in double UCB recipients^{15,16} which increase over time after transplantation¹⁷. Therefore, the utility of double UCB transplantation has remained an open question, and the origin of the unequal engraftment observed remains unresolved.

In this study, we have shown in the NOD/SCID model that mixed transplantation of two allogenic UCB grafts in the form of total MNCs leads to one-donor predominance independent of the degree of HLA-matching between the two grafts.

While the mechanisms for this unequal engraftment has not been fully elucidated, it is unlikely to originate from differences in the input CRU content of two grafts as this phenomenon is observed even when each unit of UCB showed comparable levels of engraftment as a single unit control. Moreover, in our clinical study of double cord transplantation in patients with chronic myelogenous leukemia, reversion between the dominant and non-dominant part donor was not seen in any multi-time point analysis up to 66 days after transplantation (data not shown), suggesting that the clonal heterogeneity in HSCs that exhibit kinetic differences in their clonal contribution to repopulation over the time of analysis may not be the reason for one-donor predominance.^{43,44}

In contrast, removal of lineage positive cells before grafting resulted in significant alleviation of the dominance with more balanced coengraftment, implicating that immunological competition between the grafts may occur during the engrafting process. The possibility of this immune reaction in NOD/SCID mice, despite their multiple defects in immune function⁴⁵, is supported by recent reports which have demonstrated that functional human T-cells can home and engraft in NOD/SCID mice ^{46,47} and that functional B cells⁴⁸ or dendritic cells⁴⁹ can develop after transplantation of human cord blood CD34+ cells. Consistent with these findings, infusion of human cytotoxic T-cells into tumor bearing NOD/SCID mice resulted in tumor cell killing, suggesting that human immune function can be reproduced to a certain extent in the NOD/SCID model⁵⁰.

In addition, while immune cells in UCB are relatively immature ^{12,13,51}, GVHD remains a common, albeit less severe, occurrence after UCB transplantation^{4,5}, supporting the possibility of a graft vs. graft immune reaction between the cord blood cells.

Interestingly, MSCs have been implicated in the inhibition of lymphocyte proliferation in response to mitogenic or antigenic stimuli, as well as in the inhibition of stimulated T-cells, regardless of the origin of the lymphocytes, suggesting that MSCs could exert a potent suppressive effect on the allogenic immune response^{36,40}. Taking advantage of their immune suppressive effects as well as the ease of their ex vivo expansion, we have shown that the graft vs. graft reaction which occurs in the context of double UCB transplantation can be suppressed by MSC cotransplantation, with significant alleviation of one-donor predominance.

Notably, suppressing one-donor predominance appears to be important in achieving high-level overall engraftment after double cord transplantation. When cells from one donor predominated in the recipients, as was the case for total MNC double cord transplantation, no significant improvement in the overall human cell engraftment level was achieved by such doubling of the input cell dose as compared to their single unit controls. Moreover, our multiple cohorts of double cord transplants performed with MSC cotransplantation showed significantly higher engraftment levels, and these higher levels were well correlated with more balanced coengraftment and displayed multipotent lympho-myeloid reconstitution. Taken together, these results suggest that the higher engraftment levels achieved with MSC cotransplantation are due to the more balanced coengraftment of the two allogenic cord blood cells, allowing a contribution by HSCs from both donor grafts.

Recently, Noort et al. reported that cotransplantation of cultured MSCs promoted hematopoietic engraftment despite the lack of homing by MSCs to the bone marrow⁵². We too did not find evidence for hematopoietic engraftment of MSCs by flow cytometric or genomic STR analysis. However, in our model, no significant increase in the level of engraftment was seen in the single unit controls co-transplanted with MSCs at the doses tested (data not shown). Therefore, the increase in the overall engraftment levels achieved in our double cord transplantation with MSCs would be less likely due to a direct engraftment-promoting effect of MSCs. The reason for this discrepancy is not clear, yet contributing factors could include differences in cell types and ratios of MSCs to UCB CD34⁺ cells cotransplanted. In the study by Noort et al., 1×10⁶ fetal lung-derived MSCs were cotransplanted with 0.03 to 1.0×10⁶ UCB CD34⁺ cells, and an MSC-mediated increase of UCB engraftment was seen only at a 10- to 33-fold excess of MSC over UCB CD34⁺ cells. In our model, only 4×10⁴ MSCs were infused (adjusted to be between 1 to 2×10⁶ cells/kg⁵³), and higher numbers of UCB CD34⁺ cells were co-transplanted, which could explain our lack of MSC-mediated increase in engraftment in this context.

Of note, MSCs express low levels of class 11 antigens and do not express costimulatory molecules such as B7-1, B7-2, or CD40^{26,29,40}, hence MHC-mismatched MSCs have been well tolerated in animal models^{53,54}. Furthermore, in our study, the suppressive effects of MSCs were present even for the pairs of UCBs with five HLA mismatches, raising the possibility that a greater extent of HLA disparity in UCB transplantation could be tolerated in the presence of MSCs, potentially extending the size of the donor pool among units matching in ABO blood type. Taken together, the finding that culture-expanded third-party MSCs can suppress one-donor predominance may have clinical advantages. For instance, after expanding the MSCs from third-party healthy volunteers to a large quantity in culture, aliquots of these cells could be co-transplanted with many sets of double cord transplants regardless of the donor origin. In support of this possibility, LeBlanc et al.³⁹ recently reported that MSCs could modulate mixed lymphocyte reactions independent of MHC matching status.

At present, it is not clear how MSCs, despite the fact that they do not home to bone marrow ^{52,55,56}, promote coengraftment of two UCB grafts. However, it has been known that HSC transplantation can induce donor-specific tolerance during bone marrow regeneration⁵⁷⁻⁵⁹, with mechanisms involving positive and negative T-cell selection by HSCs themselves⁶⁰, or veto cell activities of CD34+ cells^{61,62}. Therefore, a hypothesis could be put forward wherein the innate lymphocytes contained in the cord graft are suppressed by the co-transplanted MSCs, and tolerance thereafter is maintained by the HSCs from both donors (FIG. 5). However, further studies as to the effect of MSCs on other immune cells, such as natural killer cells or dendritic cells,^{63,64} are also warranted.

Although xenotransplantation into NOD/SCID mice is a popular surrogate animal model for human HSCs, certain differences between this animal model and clinical situations should be considered. First, while adoptive transfer is feasible in this model⁵⁰, the cellular nature or activities of immune cells that can be reconstituted in these mice may be different from that in a clinical setting. For example, while GVHD can occur in mice transplanted with human cells, a host vs. graft immune reaction is not operating in NOD/SCID mice. Under normal.immune conditions, however, one might expect that the MHC-independent suppressive effect of MSCs³⁶⁴⁰ may, in turn, inhibit the host immune reaction against the graft, including the possible biased immune responses toward one of the two grafts in double UCB transplantation. In support of this possibility, MSCs infused into baboons could inhibit the allogenic immune response in-vivo, increasing graft survival³⁶. Secondly, the spectrum of cellular engraftment would be different from the clinical model. Recent studies on NOD/SCID-β2M^{-/- 41} or NOD/SCID-?_{c}null mice⁶⁵ revealed that NOD/SCID mice exhibit relative difficulty in engrafting short-term repopulating cells or, in reconstituting a complete spectrum of immune cells, respectively, preferentially reflecting behaviors of the long-term repopulating cells. Furthermore, a recent gene marking study suggests that distinct HSC clones may be responsible for hematopoietic reconstitution in NOD/SCID mice versus non-human primates.⁶⁶ These results suggest that animal models more closely reflecting human hematopoiesis would better provide further insight into the clinical application of MSCs in double UCB transplantation.

The present study indicates that the one-donor predominance observed after double cord transplantation may be due to immunological competition during the in-vivo engrafting process, and that suppression by cotransplantation of the culture-expanded third-party MSCS leads to a concomitant increases in overall engraftment levels. Further studies on the long-term kinetics of MSC-mediated coengraftment as well as on the mechanisms for donor deviation should open the horizons for efficient multi-donor UCB transplantation in more severe clinical situations.

Delivery Systems

Various delivery systems are known and can be used to administer a compound of the invention, e.g., encapsulation in liposomes, microparticles, microcapsules, recombinant cells capable of expressing the compound, receptor-mediated endocytosis, construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody or a peptide of the invention, care must be taken to use materials to which the protein does not absorb. In another embodiment, the compound or composition can be delivered in a vesicle, in particular a liposome. In yet another embodiment, the compound or composition can be delivered in a controlled release system. In one embodiment, a pump may be used. In another embodiment, polymeric materials can be used. In yet another embodiment, a controlled release system can be placed in proximity of the therapeutic target, i.e., the brain, thus requiring only a fraction of the systemic dose.

A composition is said to be “pharmacologically or physiologically acceptable” if its administration can be tolerated by a recipient animal and is otherwise suitable for administration to that animal. Such an agent is said to be administered in a “therapeutically effective amount” if the amount administered is physiologically significant. An agent is physiologically significant if its presence results in a detectable change in the physiology of a recipient patient.

Kit

The invention also includes a kit or a container that includes instructions to perform stem cell cotransplantation using stem cells from multiple sources in combination with mesenchymal stromal cells. In another aspect, the invention is also directed to the written instructions per se that instruct the user to cotransplant stem cells from multiple sources and mesenchymal stromal cells. The instructions may be without limitation a label on a container or a stem cell transplantation procedure manual. Such container may be a container for a blood sample, stem cell sample, mesenchymal stromal cell sample, or any other reagent or device used in stem cell transplantation. The instructions may be via a computer screen via cathode ray tube, LCD, LED, and so on, so long as the instructions are visible through the eye. The instructions may also be in the form of audio/visual media.

The present invention is not to be limited in scope by the specific embodiments described herein. Indeed, various modifications of the invention in addition to those described herein will become apparent to theose skilled in the art from the foregoing description and accompanying figures. Such modifications are intended to fall within the scope of the appended claims. The following examples are offered by way of illustration of the present invention, and not by way of limitation.

### EXAMPLES

### Example 1

### Materials and Methods

Cells

Informed consent was obtained prior to collection of all cellular products. Low-density (1.077 g/mL) cells were isolated by Ficoll-Hypaque density centrifugation (Amersham Pharmarcia, Uppsala, Sweden) from normal cord blood samples, and cryopreserved in medium containing dimethyl sulfoxide. In some experiments, low-density cord blood cells were obtained from the buffy coats that had been cryopreserved for allogenic cord blood banking (Histostem Corp, Seoul, Korea). In all cases, HLA typing was performed by genetic typing of the HLA-A, B, and DR loci ²⁴. For lineage depletion of cord blood cells, cryopreserved light-density cells were thawed and mature lineage-positive (lin⁺) cells (CD2, CD3, CD14, CD16, CD19, CD24, CD36, CD38, CD45RA, CD56, CD66b, and glycophorin A) were depleted using an immunomagnetic column (StemCell Technologies, Vancouver, BC, Canada).

Mesenchymal stromal cells (MSCs) were obtained from normal donor bone marrow by culturing in Dulbecco's Modified Eagle's Medium (DMEM) containing 10% FBS (StemCell Technologies) and 1% PenStrep (Gibco-BRL, Rockville, Md.) as previously described²⁵, which is incorporated by reference herein in its entirety, especially as it relates to its disclosure of the characteristics of mesenchymal stromal cells. After 2 weeks of culture, adherent cells were subcultured for expansion up to three passages and cryopreserved for cotransplantation with UCB. Multi-lineage differentiation of MSCs into adipogenic, osteogenic and neurogenic lineages was confirmed by oil red-o staining, alkaline phosphatase and Alizarine-red staining, and immunohistochemical staining against NeuN, respectively, as well as by their characteristic surface marker expression as described previously. ²⁵⁻²⁹

Xenotransplantation of Cord Blood

NOD/LtSz-scid/scid (NOD/SCID) mice ²⁰, originally obtained from Dr. L. Schultz (The Jackson Laboratory, Bar Harbor, Me.), were bred and maintained in the animal facility of the Catholic Research Institutes of Medical Science (Seoul, Korea) under sterile conditions in micro-isolator cages located in an air-filtered, positively pressured room. The animals were provided with autoclaved food and water. Transplantation of cord blood cells into NOD/SCID mice was performed as previously described³⁰. Briefly, the mice received total body irradiation with 350 cGy of X-ray from a linear accelerator at 8 to 12 weeks of age, and acidified drinking water supplemented with 100 mg/L ciprofloxacin (BayerAG, Leverkusen, Germany) was provided during the experimental period. Test cells were injected intravenously into the irradiated mice within 24 hours after irradiation, and mice were sacrificed 6 weeks after transplantation. Aliquots of harvested cells were then incubated with 5% human serum and 2.4G2 (an anti-mouse Fc receptor antibody) to decrease nonspecific antibody binding.³¹ Cells were then stained for 30 minutes at 4° C. with anti-human CD45-PE (BD Pharmingen) and anti-human CD71-PE antibodies (BD Pharmingen) and washed twice in HEPES with 2% FBS; the last wash contained 1 μg/ml PI (Sigma Chemical, St. Louis, Mo.). A detection limit of more than 1% human cells among the total PI⁻ cells was used to identify positively engrafted mice using gates set to exclude more than 99.99% of nonspecifically stained PI⁻ cells incubated with isotype-matched control antibodies labeled with the corresponding fluorochromes, as previously described. Genomic DNA was purified from aliquots of bone marrow obtained from animals engrafted with human cells using a QI Amp® DNA Mini kit (QIAGEN, Hilden, Germany) and subjected to analysis for donor origin.

Mixed Transplantation of Cord Blood

Pairs of cord blood units with varying degrees of HLA-disparities were selected from the pool of cord blood that had been HLA typed at the time of freezing. Light density cells from these cord blood units were stained with anti-CD34-FITC (BD Pharmingen, San Diego, Calif.) and anti-CD3-FITC (BD Pharmingen) to determine CD34⁺ and CD3⁺ cell content. Cells from each single cord unit were aliquoted to contain equivalent numbers of CD34⁺ cell at limiting dose (3-5×10⁴ CD34⁺ cells/mouse) and transplanted into irradiated NOD/SCID mice both as a single unit, or as part of double unit transplantation, so that relative contribution by each donor cells could be analyzed on the basis of input HSCs transplanted. Donor origin of the engrafted cells in the NOD/SCID mice was analyzed either by PCR-SSOP or by real-time quantitative PCR of 16 STR markers (RQ-STR).

Qualitative and Quantitative Analysis of Donor Origin of Engrafted Cells

PCR-SSOP (sequence-specific oligonucleotide probe) was used to type HLA-DRB1, DQA1, DQB1, and DPB1 loci in addition to the HLA-A, B, DR loci to analyze the donor distribution of engrafted cells as previously described,²⁴ with minor modifications. Each HLA-locus gene was amplified by specific primers, and the products denatured and immobilized on a nylon membrane for probing with digoxigenin-labeled oligonucleotide probes specific for known hypervariable sequences. Stringent washing was performed in the presence of tetramethyl ammonium chloride (TMAC, Sigma Chemical). The hybridized filters were then probed with an alkaline phosphatase-conjugated anti-digoxigenin antibody, and visualized with chemiluminiscent substrate CSPD (Boehringer Mannheim, GmbH, Germany).

For quantitative analysis of donor distribution in engrafted cells, multiplex real-time quantitative PCR (RQ-PCR) on human short tandem repeats (STR) was performed as previously described 32 using the AmpFISTR Identifier PCR Amplification Kit (PE Applied Biosystems, Foster City, Calif.). The following 16 STR markers were amplified in this system: D8S1179, D21S11, D7S820, CSFLPO, D3S1358, TH01, D13S317, D16S539, D2S1338, D19S433, vWA, TPOX, D18S51, D5S818, FGA, and the gender marker amelogenin. All markers were amplified in a multiplex PCR reaction (PCRExpress, HYBAID, Ashford, Middlesex, UK) according to the manufacturer's instructions, and the resulting fragments were analyzed on the ABI Prism 310 Genetic Analyzer (PE Applied Biosystems). 1.5 μl of the PCR product was added to 24.5 μl of Hi-Di formamide (PE Applied Biosystems) and 0.5 μl of GeneScan-500 LIZ Size Standard (PE Applied Biosystems), and then subjected to capillary electrophoresis using Performance Optimized Polymer (PE Applied Biosystems) with a 47 cm/50 μm capillary (PE Applied Biosystems). Fragment sizes and peak areas were analyzed by the GeneScan Analyzer and Genotyper software (PE Applied Biosystems).

The extent of double chimerism was calculated from the observed peak area of the informative markers. Only informative alleles including type 1 (non-overlapping) and type 11 (partial overlapping) were taken for calculation of chimerism with exclusion of type 111 (overlapping) alleles.

Statistical Analyses

The results are shown as mean values ±SEM from independent experiments. Differences between groups were analyzed using the Student t-test.

### Example 2

### Results

Mixed Transplantation of Total Nucleated Cells Leads to One-Donor Predominance Independent of Input CRU Content or Degree of HLA Mismatch.

Light density total mononuclear cells (MNC) from pairs of UCB units having differing degrees of HLA-disparity were prepared from previously HLA-screened UCB pools and transplanted into NOD/SCID mice alone (control) or as a mixture. The percentage of CD34⁺ cells in these pools ranged between 0.2% and 1.0% (mean 0.6%). Although approximately 30% of mice died during the first 2 to 3 weeks after transplantation, no signs of GVHD such as shivering, hair loss, or gut necrosis 33,34 were observed. Mice were sacrificed 6 weeks after transplantation and analyzed for human cell engraftment and donor origin of the engrafted cells.

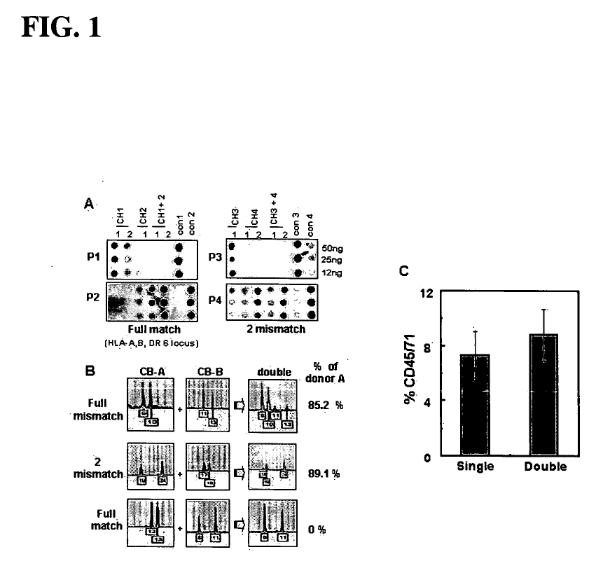

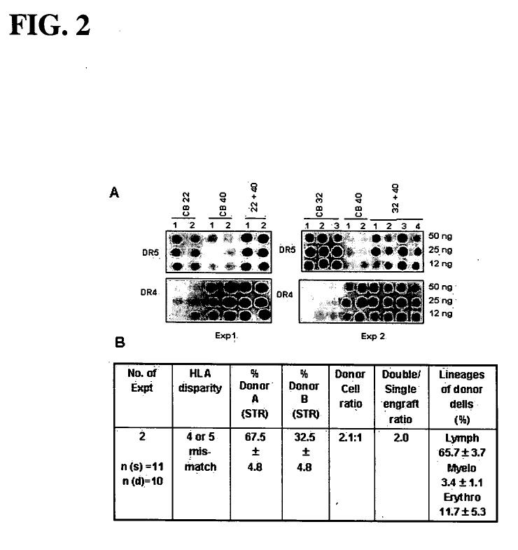

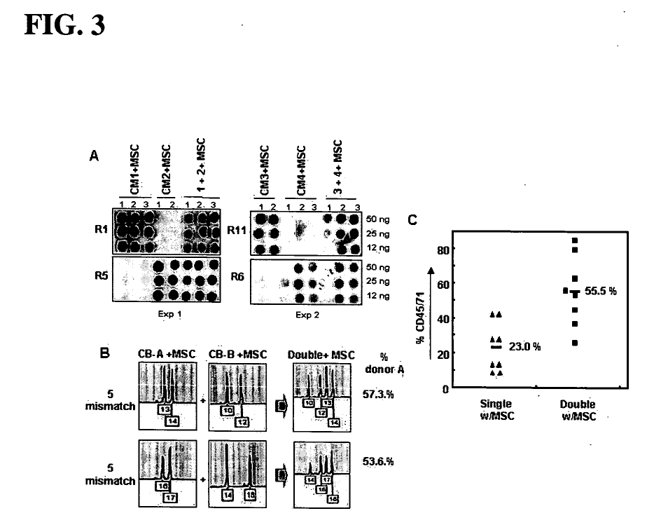

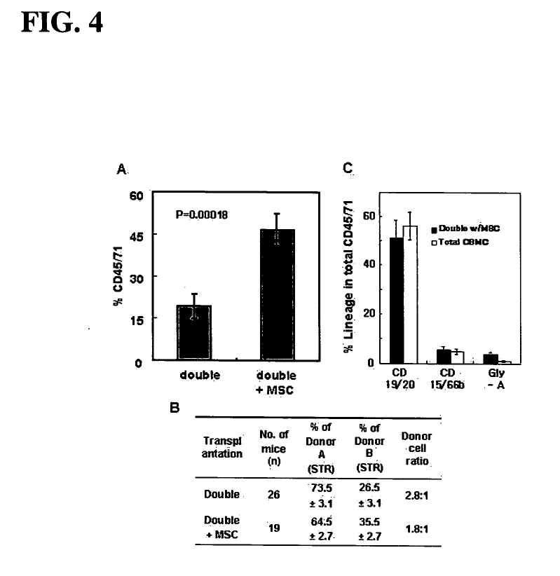

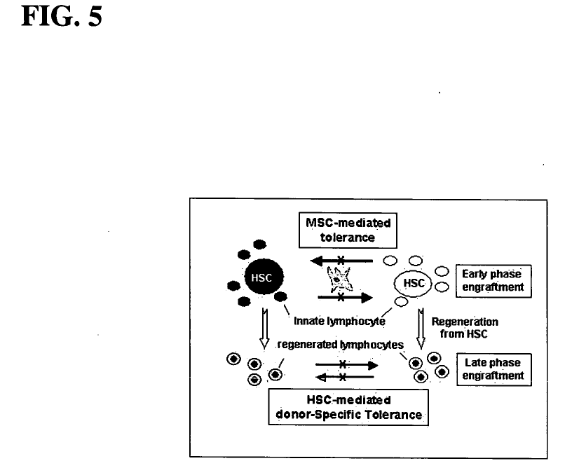

First, three cohorts of double cord transplants with either full matches in six loci (HLA-A, B, DR in each haploid), two mismatches (in HLA-B, DR) or full mismatches were analyzed with PCR-SSOP for the relative contribution of each donor and compared to each matched single unit control. As shown in FIG. 1A, transplantation of each UCB as a single graft resulted in a readily detectable and comparable level human cell engraftment in the mice (CH1 to CH4) as evidenced by the density of hybridization of each allele-specific oligonucleotide probe (P1 to P4). However, in the mixed transplantation of two units (CH1+2, or CH3+4), only a single probe (P2 specific for CH2 or P4 specific for CH4) hybridized to DNA obtained from the recipients' bone marrow and no specific hybridization signal was detected from the other unit (P1 for CH1, or P3 for CH3). Double transplantation of two fully mismatched units also demonstrated a similar one-donor predominance (data not shown). These results indicate that one-donor predominance can occur in double transplantation of total MNCs even when each unit of UCB can engraft at comparable levels when infused as a single graft, suggesting that this phenomenon is not due to a lack of input CRU in the non-dominant graft.

To quantitatively measure the extent and variability of one-donor predominance with respect to HLA-disparity, we next performed RQ-PCR of donor-specific STR and analyzed the distribution of donor engraftment from 9 cohorts of double cord transplant recipients receiving grafts with varying degrees of HLA-disparity (summarized in Table 1). FIG. 1B illustrates a representative analysis, where STR peaks derived from the dominant party constitute 85% to 100% of donor-derived peaks amplified. Table 1 summarizes the quantitation from each HLA-matching category. As shown, minor variations in the extent of one-donor predominance were observed within each HLA-matching group, but no significant difference in the extent or frequency of one-donor predominance was observed between the various HLA-matching groups, with overall dominant cells comprising 80.74±2.2% of engraftment in recipient animals (average donor cell ratio of 4.2:1). Additionally, none of the parameters, including CD34⁺ cell percentage, CD3⁺ cell percentage, or presence of a particular HLA type correlated with the dominance observed.

TABLE 1
Cumulative quantitation of donor cell distribution using RQ-STR
analysis in double cord transplantation of total mononuclear cells

| HLA | No. of Exp | % Donor | % Donor | | No of mice |
|---|---|---|---|---|---|
| disparity (no. of | A | B | Donor cell | with | |
| (A, B, DR) | mice) | (STR) | (STR) | ratio | predominance |
| Full match | 3 | 82.8 ± 7.1 | 17.2 ± 7.1 | 4.8:1 | 4/5 |
| | (n = 5) | | | | |
| 1 mismatch | 2 | 83.8 ± 2.2 | 16.2 ± 2.2 | 5.2:1 | 8/8 |
| | (n = 8) | | | | |
| 2 mismatch | 2 | 74.3 ± 5.6 | 25.7 ± 5.6 | 2.9:1 | 3/5 |
| | (n = 5) | | | | |
| Full mismatch | 2 | 80.6 ± 3.5 | 19.4 ± 3.5 | 4.2:1 | 3/4 |
| | (n = 4) | | | | |
| Total | 9 | 80.7 ± 2.2 | 19.3 ± 2.2 | 4.2:1 | 18/22 |
| | (n = 22) | | | | |

Total MNCs equivalent to 5 × 10⁴ CD34⁺ cells from each UCB unit were mixed and transplanted. The percentage of donor A (dominant party) and donor B (non-dominant party) were determined by measuring the area of donor-specific STR peaks among all human-specific STR peaks, and the donor A to donor B cell ratio calculated. Ratios greater than 3:1 were considered dominant and the mice scored as donor predominant.

We next compared the overall engraftment levels achieved after double cord transplantation to that achieved after single unit transplantation. As shown in FIG. 1C, transplantation of two UCB units as a mixture of total MNCs led to overall human cell engraftment ranging from 2.2% to 31.0% (mean 8.8+1.9 %), while transplantation of matched single UCB units led to engraftment of 1.1% to 33.1% (mean 7.3+1.8 %), thus showing no significant difference in the level of engraftment between single and double cord transplantation, despite the fact that twice as many cells were transplanted for the latter. This result suggests that, under circumstances of one-donor predominance, mixing two cord units does not lead to a significant increase in the overall engraftment level.

Lineage Depletion of the Graft Alleviates One-Donor Predominance

To determine whether one-donor predominance can be attributed to an in vivo graft versus graft reaction between the two allogenic cord blood grafts, four cohorts of double cord transplants were performed after depletion of lineage positive cells and the relative distribution of donor derived cells compared.

In the first two experiments analyzed by PCR-SSOP, two pairs of UCB with either five (CB32, CB40) or six mismatches (CB22, CB40) were double transplanted, with corresponding single unit grafts transplanted in parallel. As shown (FIG. 2A), despite a high degree of mismatch, remarkable coengraftment levels were observed in the double transplant recipients (22+40, 32+40), as evidenced by comparable intensity of hybridization by each donor-specific probe (DR4 to DR6).

Quantitative analysis of the donor cell engraftment ratios from two additional cohorts also showed similar alleviation of the one-donor predominance, with dominant donor cells comprising only 67.5±4.8% (donor cell ratio, 2.1:1) (FIG. 2B). Moreover, the engraftment levels observed after lineage-depleted double UCB transplants were 2-fold higher than that after single UCB transplants, with both lymphoid and myeloid reconstitution.

Taken together, these results show that depletion of lineage-positive cells from double UCB grafts can lead to improved donor coengraftment, and that a graft vs. graft reaction with immunological competition may play a role in the process of one-donor predominance.

Cotransplantation of Third-Party MSC can Alleviate One-Donor Predominance and Result in Additive Coengraftment

Although lineage depletion alleviates one-donor predominance, alternative strategies not requiring lineage depletion would be more desirable as the latter is frequently associated with the cell loss³⁵, which might itself hamper reaching the target cell dose. To circumvent this hurdle, we postulated that cotransplantation of mesenchymal stromal cells (MSCs), bone marrow-derived cells that have been implicated in the suppression of allogenic immune response³⁶⁻⁴⁰, could also suppress the graft vs. graft reaction in double cord transplantation.

Therefore, MSC cultures were established from third-party bone marrow, and expanded in culture up to three passages, a passage number at which these cells retain their phenotypic characteristics and multi-lineage differentiation potential as previously defined²⁹ (data not shown). Aliquots of expanded MSCs were then co-transplanted with total MNCs from each UCB unit as part of double or single-unit transplant to examine the effects on donor cell distribution. In the first two experiments using two pairs of UCB units with 5 mismatches each, remarkable degrees of coengraftment by each donor unit were achieved in the mixed transplants (1+2+MSC and 3+4+MSC), as evidenced by comparable intensities of hybridization by each donor-specific probe, which were also proportional to the intensities seen in the recipients of single unit controls (CM1+MSC to CM4+MSC) (FIG. 3A).

RQ-STR analysis performed on these cohorts with MSC cotransplantation also showed that cells from both donors made a comparable contribution to bone marrow reconstitution, as evidenced by the coexistence of each donor-specific STR peak at similar amplitudes (FIG. 3B). Cumulative measurement of the donor distribution showed that the dominant cells comprised 66.5±4.4% of engraftment, with a donor cell ratio of 2.0:1 (Table 2), which was significantly lower than that observed after double cord transplantation without MSC cotransplantation (4.2:1) (Table 1).

TABLE 2
Cumulative quantitation of donor cell distribution using RQ-STR
analysis in double cord transplantation with MSC co-infusion

| | % of Donor A | % of Donor B | Donor cell |
|---|---|---|---|
| STR | (STR) | (STR) | ratio |
| Type I | 66.7 ± 4.4 | 33.3 ± 4.4 | 2.0:1 |
| Type II | 63.7 ± 3.9 | 36.3 ± 3.9 | 1.8:1 |
| Total | 66.5 ± 4.4 | 33.5 ± 4.4 | 2.0:1 |

n =8; from cohorts that each included four pairs of UCB units: one three-mismatch pair, two five-mismatch pairs, and one full-mismatch pair. Mononuclear cells equivalent to 3 × 10⁴ CD34+ cells for each UCB were mixed and transplanted with 4 × 10⁴ cultured MSCS. The percentage of donor specific contribution and the donor cell ratio were calculated as described.

Of note, in the MSC co-transplanted cohorts, while single cord blood transplantation showed an average of 23.0+4.6% (n=8) overall engraftment, double cord blood transplantation showed 55.5+6.7% (n=8), a nearly two-fold increase(FIG. 3C). This increased level of engraftment after double cord transplantation contrasts sharply to that achieved without MSC cotransplantation (FIG. 1), where no significant difference in the overall engraftment level was observed between the single and double cord blood transplants.

Higher-Level Engraftment and Balanced Coengraftment can be Achieved in Double Cord Transplantation with MSC Cotransplantation

In order to determine whether cotransplantation of MSCs could indeed bring about a beneficial outcome in double cord transplantation, we directly compared multiple independent cohorts of double cord transplants in the presence or absence of MSCs (FIG. 4). As shown in FIG. 4A, while transplantation of double cord units (equivalent to 3×10⁴ CD34+ cells each) without MSCs showed 19.1+4.4% total human cell engraftment (n=26), those with MSC cotransplantation showed 46.6±5.8% (n=19), demonstrating a significantly higher level of engraftment with MSC cotransplantation (P=0.00018).

Notably, the higher level engraftment observed with MSC cotransplantation correlated with alleviation of one-donor predominance (FIG. 4B), i.e., while the dominant unit represented 73.5% (donor cell ratio 2.8:1) of the engrafted cells in conventional double cord transplantation without MSC, it was reduced to 64.5% (donor ratio 1.8:1) with MSC cotransplantation, showing more balanced coengraftment. Additionally, no significant difference in the lineage distribution of engrafted cells was seen in the presence or absence of MSC cotransplantation (FIG. 4C), precluding the possibility that the increase in engraftment level with MSC cotransplantation was produced by distinct HSC populations with short-term, lineage restricted potentials.

Taken together, these results show that cotransplantation of culture-expanded third-party MSCs results in higher level engraftment after double cord transplantation, and that such increased engraftment can be partly, if not completely, attributed to a reduced extent of donor deviation between the two grafts. Further, these results demonstrate the importance of alleviating one-donor predominance as a means to improve outcome after double cord transplantation.

References

1. Holyoake T L, Nicolini F E, Eaves C J. Functional differences between transplantable human hematopoietic stem cells from fetal liver, cord blood, and adult marrow. Exp Hematol. 1999;27:1418-1427
2. Wang J C, Doedens M, Dick J E. Primitive human hematopoietic cells are enriched in cord blood compared with adult bone marrow or mobilized peripheral blood as measured by the quantitative in vivo SCID-repopulating cell assay. Blood. 1997;89:3919-3924.
3. Cairo M S, Wagner J E. Placental and/or umbilical cord blood: an alternative source of hematopoietic stem cells for transplantation. Blood. 1997;90:4665-4678.
4. Gluckman E, Rocha V, Boyer-Chammard A, et al. Outcome of cord-blood transplantation from related and unrelated donors. Eurocord Transplant Group and the European Blood and Marrow Transplantation Group. N Engl J Med. 1997;337:373-381.
5. Rubinstein P, Carrier C, Scaradavou A, et al. Outcomes among 562 recipients of placental-blood transplants from unrelated donors. N Engl J Med. 1998;339:1565-1577.
6. Sekhar M, Yu J M, Soma T, Dunbar C E. Murine long-term repopulating ability is compromised by ex vivo culture in serum-free medium despite preservation of committed progenitors. J Hematother. 1997;6:543-549.
7. McNiece I K, Almeida-Porada G, Shpall E J, Zanjani E. Ex vivo expanded cord blood cells provide rapid engraftment in fetal sheep but lack long-term engrafting potential. Exp Hematol. 2002;30:612-616.
8. Rebel V I, Tanaka M, Lee J S, et al. One-day ex vivo culture allows effective gene transfer into human nonobese diabetic/severe combined immune-deficient repopulating cells using high-titer vesicular stomatitis virus G protein pseudotyped retrovirus. Blood. 1999;93:2217-2224.
9. Dorrell C, Gan O I, Pereira D S, Hawley R G, Dick J E. Expansion of human cord blood CD34(+)CD38(−) cells in ex vivo culture during retroviral transduction without a corresponding increase in SCID repopulating cell (SRC) frequency: dissociation of SRC phenotype and function. Blood. 2000;95:102-110.
10. Jaroscak J, Goltry K, Smith A, et al. Augmentation of umbilical cord blood (Ucb) transplantation with ex-vivo expanded UCB cells: results of a phase I trial using the Aastrom Replicell system. Blood. 2003;101:5061-5067.
11. Traycoff C M, Orazi A, Ladd A C, Rice S, McMahel J, Srour E F. Proliferation induced decline of primitive hematopoietic progenitor cell activity is coupled with an increase in apoptosis of ex vivo expanded CD34+ cells. Exp Hematol. 1998;26:53-62.
12. Hunt D W, Huppertz H I, Jiang H J, Petty R E. Studies of human cord blood dendritic cells: evidence for functional immaturity. Blood. 1994;84:4333-4343.
13. Nomura A, Takada H, Jin C H, Tanaka T, Ohga S, Hara T. Functional analyses of cord blood natural killer cells and T cells: a distinctive interleukin-18 response. Exp Hematol. 2001;29:1169-1176.
14. Rocha V, Wagner J E, Jr., Sobocinski K A, et al. Graft-versus-host disease in children who have received a cord-blood or bone marrow transplant from an HLA-identical sibling. Eurocord and International Bone Marrow Transplant Registry Working Committee on Alternative Donor and Stem Cell Sources. N Engl J Med. 2000;342:1846-1854.
15. Weinreb S, Delgado J C, Clavijo O P, et al. Transplantation of unrelated cord blood cells. Bone Marrow Transplant. 1998;22:193-196.
16. Barker J N, Weisdorf D J, Wagner J E. Creation of a double chimera after the transplantation of umbilical-cord blood from two partially matched unrelated donors. N Engl J Med. 2001;344:1870-1871.
17. Barker J N, Weisdorf D J, DeFor T E, McGlave P B, Wagner J E. Multiple unit unrelated donor umbilical cord blood transplantation in high risk adults with hematologic malignancies: Impact on engraftment and chimerism[abstract]. Blood. 2002;100:41a
18. De Lima M, St John L S, Wieder E D, et al. Double-chimaerism after transplantation of two human leucocyte antigen mismatched, unrelated cord blood units. Br J Haematol. 2002;119:773-776.
19. McCune J M, Namikawa R, Kaneshima H, Shultz L D, Lieberman M, Weissman I L. The SCID-hu mouse: murine model for the analysis of human hematolymphoid differentiation and function. Science. 1988;241:1632-1639.
20. Larochelle A, Vormoor J, Hanenberg H, et al. Identification of primitive human hematopoietic cells capable of repopulating NOD/SCID mouse bone marrow: implications for gene therapy. Nat Med. 1996;2:1329-1337.
21. Civin C I, Almeida-Porada G, Lee M J, Olweus J, Terstappen L W, Zanjani E D. Sustained, retransplantable, multilineage engraftment of highly purified adult human bone marrow stem cells in vivo. Blood. 1996;88:4102-4109.
22. Shi P A, Hematti P, von Kalle C, Dunbar C E. Genetic marking as an approach to studying in vivo hematopoiesis: progress in the non-human primate model. Oncogene. 2002;21:3274-3283.
23. de Pauw E S, Otto S A, Wijnen J T, et al. Long-term follow-up of recipients of allogeneic bone marrow grafts reveals no progressive telomere shortening and provides no evidence for haematopoietic stem cell exhaustion. Br J Haematol. 2002;116:491-496.
24. Jordan F, McWhinnie A J, Turner S, et al. Comparison of HLA-DRB1 typing by DNA-RFLP, PCR-SSO and PCR-SSP methods and their application in providing matched unrelated donors for bone marrow transplantation. Tissue Antigens. 1995;45:103-110.
25. Prockop D J. Marrow stromal cells as stem cells for nonhematopoietic tissues. Science. 1997;276:71-74.
26. Pittenger M F, Mackay A M, Beck S C, et al. Multilineage potential of adult human mesenchymal stem cells. Science. 1999;284:143-147.
27. Mackay A M, Beck S C, Murphy J M, Barry F P, Chichester C O, Pittenger M F. Chondrogenic differentiation of cultured human mesenchymal stem cells from marrow. Tissue Eng. 1998;4:415-428.
28. Woodbury D, Schwarz E J, Prockop D J, Black I B. Adult rat and human bone marrow stromal cells differentiate into neurons. J Neurosci Res. 2000;61:364-370.
29. Deans R J, Moseley A B. Mesenchymal stem cells: biology and potential clinical uses. Exp Hematol. 2000;28:875-884.
30. Glimm H, Oh I H, Eaves C J. Human hematopoietic stem cells stimulated to proliferate in vitro lose engraftment potential during their S/G(2)/M transit and do not reenter G(0). Blood. 2000;96:4185-4193.
31. Unkeless J C. Characterization of a monoclonal antibody directed against mouse macrophage and lymphocyte Fc receptors. J Exp Med. 1979;150:580-596.
32. Kim Y J, Kim D W, Lee S, et al. Comprehensive comparison of FISH, RT-PCR, and RQ-PCR for monitoring the BCR-ABL gene after hematopoietic stem cell transplantation in CML. Eur J Haematol. 2002;68:272-280.
33. Takatsuka H. Iwasaki T, Okamoto T, Kakishita E. Intestinal graft-versus-host disease: mechanisms and management. Drugs. 2003;63:1-15.
34. Hill G R, Ferrara J L. The primacy of the gastrointestinal tract as a target organ of acute graft-versus-host disease: rationale for the use of cytokine shields in allogeneic bone marrow transplantation. Blood. 2000;95:2754-2759.
35. Johnsen HE, Hutchings M, Taaning E, et al. Selective loss of progenitor subsets following clinical CD34+ cell enrichment by magnetic field, magnetic beads or chromatography separation. Bone Marrow Transplant. 1999;24:1329-1336.
36. Bartholomew A, Sturgeon C, Siatskas M, et al. Mesenchymal stem cells suppress lymphocyte proliferation in vitro and prolong skin graft survival in vivo. Exp Hematol. 2002;30:42-48.
37. Di Nicola M, Carlo-Stella C, Magni M, et al. Human bone marrow stromal cells suppress T-lymphocyte proliferation induced by cellular or nonspecific mitogenic stimuli. Blood. 2002;99:3838-3843.
38. Krampera M, Glennie S, Dyson J, et al. Bone marrow mesenchymal stem cells inhibit the response of naive and memory antigen-specific T cells to their cognate peptide. Blood. 2002;101:3722-3729
39. Le Blanc K, Tammik L, Sundberg B, Haynesworth S E, Ringden 0. Mesenchymal stem cells inhibit and stimulate mixed lymphocyte cultures and mitogenic responses independently of the major histocompatibility complex. Scand J Immunol. 2003;57:11-20.
40. Tse W T, Pendleton J D, Beyer W M, Egalka M C, Guinan E C. Suppression of allogeneic T-cell proliferation by human marrow stromal cells: implications in transplantation. Transplantation. 2003;75:389-397.
41. Glimm H, Eisterer W, Lee K, et al. Previously undetected human hematopoietic cell populations with short-term repopulating activity selectively engraft NOD/SCID-beta2 microglobulin-null mice. J Clin Invest. 2001;107:199-206.
42. Lim F, Beckhoven J, Brand A, et al. The number of nucleated cells reflects the hematopoietic content of umbilical cord blood for transplantation. Bone Marrow Transplant. 1999;24:965-970.
43. Kim H J, Tisdale J F, Wu T, et al. Many multipotential gene-marked progenitor or stem cell clones contribute to hematopoiesis in nonhuman primates. Blood. 2000;96: 1-8.
44. Guenechea G, Gan I O, Dorrell C, Dick J E. Distinct classes of human stem cells that differ in proliferative and self-renewal potential. Nat Immunol. 2001;2:75-82.
45. Shultz L D, Schweitzer P A, Christianson S W, et al. Multiple defects in innate and adaptive immunologic function in NOD/LtSz-scid mice. J Immunol. 1995;154:180-191.
46. Di lanni M, Terenzi A, Falzetti F, et al. Homing and survival of thymidine kinase-transduced human T cells in NOD/SCID mice. Cancer Gene Ther. 2002;9:756-761.
47. Kerre T C, De Smet G, De Smedt M, et al. Adapted NOD/SCID model supports development of phenotypically and functionally mature T cells from human umbilical cord blood CD34(+) cells. Blood. 2002;99:1620-1626.
48. Li C, Ando K, Kametani Y, et al. Reconstitution of functional human B lymphocytes in NOD/SCID mice engrafted with ex vivo expanded CD34(+) cord blood cells. Exp Hematol. 2002;30:1036-1043.
49. Palucka A K, Gatlin J, Blanck J P, et al. Human dendritic cell subsets in NOD/SCID mice engrafted with CD34+ hematopoietic progenitors. Blood. 2003;17:17
50. Feuerer M, Beckhove P, Bai L, et al. Therapy of human tumors in NOD/SCID mice with patient-derived reactivated memory T cells from bone marrow. Nat Med. 2001;7:452-458.
51. Jiang Q, Azuma E, Hirayama M, et al. Functional immaturity of cord blood monocytes as detected by impaired response to hepatocyte growth factor. Pediatr Int. 2001;43:334-339.
52. Noort W A, Kruisselbrink A B, in't Anker PS, et al. Mesenchymal stem cells promote engraftment of human umbilical cord blood-derived CD34(+) cells in NOD/SCID mice. Exp Hematol. 2002;30:870-878.
53. Devine S M, Bartholomew A M, Mahmud N, et al. Mesenchymal stem cells are capable of homing to the bone marrow of non-human primates following systemic infusion. Exp Hematol. 2001;29:244-255.
54. Bartholomew A, Patil S, Mackay A, et al. Baboon mesenchymal stem cells can be genetically modified to secrete human erythropoietin in vivo. Hum Gene Ther. 2001;12:1527-1541.
55. Rombouts W J, Ploemacher R E. Primary murine MSC show highly efficient homing to the bone marrow but lose homing ability following culture. Leukemia. 2003;17:160-170.
56. Koc O N, Peters C, Aubourg P, et al. Bone marrow-derived mesenchymal stem cells remain host-derived despite successful hematopoietic engraftment after allogeneic transplantation in patients with lysosomal and peroxisomal storage diseases. Exp Hematol. 1999;27:1675-1681.
57. Charlton B, Auchincloss H, Jr., Fathman C G. Mechanisms of transplantation tolerance. Annu Rev Immunol. 1994;12:707-734.
58. Wekerle T, Sykes M. Mixed chimerism as an approach for the induction of transplantation tolerance. Transplantation. 1999;68:459-467.
59. Billingham R E, Brent, L. Medawar P. B. Actively acquired tolerance of foreign cells. Nature. 1953;172:603-606
60. Shizuru J A, Weissman I L, Kernoff R, Masek M, Scheffold Y C. Purified hematopoietic stem cell grafts induce tolerance to alloantigens and can mediate positive and negative T cell selection. Proc Natl Acad Sci U S A. 2000;97:9555-9560.
61. Gur H, Krauthgamer R, Berrebi A, et al. Tolerance induction by megadose hematopoietic progenitor cells: expansion of veto cells by short-term culture of purified human CD34(+) cells. Blood. 2002;99:4174-4181.
62. Rachamim N, Gan J, Segall H, et al. Tolerance induction by “megadose” hematopoietic transplants: donor-type human CD34 stem cells induce potent specific reduction of host anti-donor cytotoxic T lymphocyte precursors in mixed lymphocyte culture. Transplantation. 1998;65:1386-1393.
63. Goldschneider I, Cone R E. A central role for peripheral dendritic cells in the induction of acquired thymic tolerance. Trends Immunol. 2003;24:77-81.
64. Higuchi M, Zeng D, Shizuru J, et al. Immune tolerance to combined organ and bone marrow transplants after fractionated lymphoid irradiation involves regulatory NK T cells and clonal deletion. J Immunol. 2002;169:5564-5570.
65. Hiramatsu H, Nishikomori R, Heike T, et al. Complete reconstitution of human lymphocytes from cord blood CD34+ cells using the NOD/SCID/gammacnull mice model. Blood. 2003;102:873-880.
66. Horn P A, Thomasson B M, Wood B L, Andrews R G, Morris J C, Kiem H P. Distinct hematopoietic stem/progenitor cell populations are responsible for repopulating NOD/SCID mice versus nonhuman primates. Blood. 2003;19:19

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

1. A composition comprising:
(i) a sample of donor stem cells that are desired to be engrafted to a subject; and
(ii) engraftment effective amount of mesenchymal stromal cells.

2. The composition according to claim 1, wherein the sample in (i) comprises bone marrow stem cells, peripheral blood stem cells, or umbilical cord blood stem cells or a mixture thereof.

3. The composition according to claim 2, wherein the sample in (i) comprises umbilical cord blood stem cells that are allogeneic to each other.

4. The composition according to claim 1, wherein the mesenchymal stromal cells are derived from third party bone marrow or umbilical cord blood.

5. A container comprising stock composition of mesenchymal stromal cells and instructions for using the mesenchymal stromal cells in cotransplantation with donor stem cells.

6. A method for increasing engraftment of donor stem cells in a subject comprising administering the composition according to claim 1 to a subject in need thereof.

7. The method according to claim 6, wherein the sample in (i) comprises bone marrow stem cells, peripheral blood stem cells, or umbilical cord blood stem cells or a mixture thereof.

8. The method according to claim 7, wherein the sample in (i) comprises umbilical cord blood stem cells that are allogeneic to each other.

9. The method according to claim 6, wherein the donor stem cells are single unit.

10. The method according to claim 6, wherein the donor stem cells are multiple units.

11. A method for suppressing immune reaction in vivo comprising administering the composition according to claim 1 to a subject in need thereof.

12. A method for suppressing graft versus host disease comprising administering the composition according to claim 1 to a subject in need thereof.

13. The method according to claim 6, wherein the mesenchymal stromal cells are derived from third party bone marrow or umbilical cord blood.

14. The method according to claim 11, wherein the mesenchymal stromal cells are derived from third party bone marrow or umbilical cord blood.

15. The method according to claim 12, wherein the mesenchymal stromal cells are derived from third party bone marrow or umbilical cord blood.
